(19) United States
(12) Patent Application Publication
(10) Pub. No.: US 2010/0144020 A1
(43) Pub. Date: Jun. 10, 2010

(54) DISPOSABLE DIAGNOSTIC KIT

(75) Inventors: Kyung Hyun Kim, Daejeon (KR); Wanjoong Kim, Goyang-si (KR); Bong Kyu Kim, Daejeon (KR); Hyunsung Ko, Seoul (KR); Chul Huh, Daejeon (KR); Gun Yong Sung, Daejeon (KR); Seon-Hee Park, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daegeon (KR)

(21) Appl. No.: 12/421,796

(22) Filed: Apr. 10, 2009

(30) Foreign Application Priority Data
Dec. 8, 2008 (KR) 10-2008-123912

(51) Int. Cl. C12M 1/34; B01L 3/00

(52) U.S. Cl. 435/287.1; 422/61

(57) ABSTRACT

Provided is a disposable diagnostic kit capable of diagnosing diseases. The disposable diagnostic kit includes a preprocessor, a target material reactor, and a microfluidic channel. The preprocessor filters target materials from a fluid containing various biomaterials. The target material reactor includes a diffraction grating on whose surface sensing materials reacting with the target materials are immobilized. Herein, a wavelength of light penetrated into the diffraction grating or a wavelength of light reflected by the diffraction grating varies depending on the target materials. The microfluidic channel moves the filtered fluid from the preprocessor to the target material reactor.

### CROSS-REFERENCE TO RELATED APPLICATIONS

This U.S. non-provisional patent application claims priority under 35 U.S.C. §119 of Korean Patent Application No. 10-2008-0123912, filed on Dec. 8, 2008, the entire contents of which are hereby incorporated by reference.

### BACKGROUND OF THE INVENTION

The present invention disclosed herein relates to a disposable diagnostic kit, and more particularly, to a disposable diagnostic kit capable of diagnosing diseases more simply.

A lap-on-a-chip is a device capable of analyze biomaterials, in which the coupling and reaction between a sample and a reagent, the creation of reactants, and the output of physical signals corresponding to the reactants are performed in the single chip. The lap-on-a-chip is used in hospitals or homes as a disposable diagnostic kit that can rapidly diagnose diseases using a small amount of biomaterial. Examples of the disposable diagnostic kit are a home pregnancy diagnostic kit, a blood sugar diagnostic kit, and an emergency-room AIDS diagnostic kit.

The disposable diagnostic kit requires a high-resolution sensor in order to detect an extremely small amount of biomaterial, qualitative information of biomaterial, and quantitative information of biomaterial. Also, the disposable diagnostic kit uses a technology for moving a body fluid such as blood or urine a sensor, and a technology for changing the body fluid at the sensor for detection by the naked eye. The disposable diagnostic kit may also use a technology for electrochemically measuring a micro current or voltage that is generated at an electrode according to a biomaterial.

However, if various fluorescent materials, dyes, or nanoparticles are used to identify biomaterials with the naked eye, the biomaterial may be deformed by the coupling between the color-developing material and the biomaterial.

Also, in the electrochemical method, an electrode must be provided at the outside or inside of a disposable diagnostic kit in order to measure a micro current or voltage. This may complicate the fabrication process of the disposable diagnostic kit and may increase the fabrication cost. Also, because the electrode is provided at the disposable diagnostic kit, the electrical characteristics may vary depending on the storage conditions (e.g., humidity and temperature) of the disposable diagnostic kit.

### SUMMARY OF THE INVENTION

The present invention provides a disposable diagnostic kit capable of diagnosing diseases more simply.

The object of the present invention is not limited to the aforesaid, but other objects not described herein will be clearly understood by those skilled in the art from descriptions below.

Embodiments of the present invention provide disposable diagnostic kits including: a preprocessor filtering target materials from a fluid containing various biomaterials; a target material reactor comprising a diffraction grating on whose surface sensing materials reacting with the target materials are immobilized, wherein a wavelength of light penetrated into the diffraction grating or a wavelength of light reflected by the diffraction grating varies depending on the target materials; and a microfluidic channel moving the filtered fluid from the preprocessor to the target material reactor.

The details of other embodiments are included in the detailed description and the drawings.

### BRIEF DESCRIPTION OF THE FIGURES

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIG. 1 is a perspective view of a disposable diagnostic kit according to an exemplary embodiment of the present invention;

FIG. 2 is a view showing a top plate of the disposable diagnostic kit according to an exemplary embodiment of the present invention;

FIG. 3 is a view showing a bottom plate of the disposable diagnostic kit according to an exemplary embodiment of the present invention;

FIG. 4 is a cross-sectional view taken along a line I-I′ of FIG. 1; and

FIG. 5 is a view showing a target material reactor of the disposable diagnostic kit according to an exemplary embodiment of the present invention.

### DETAILED DESCRIPTION OF PREFERRED EMBODIMENTS

Preferred embodiments of the present invention will be described below in more detail with reference to the accompanying drawings. The present invention may, however, be embodied in different forms and should not be constructed as limited to the embodiments set forth herein. Rather, these embodiments are provided so that this disclosure will be thorough and complete, and will fully convey the scope of the present invention to those skilled in the art. Like reference numerals refer to like elements throughout.

In the following description, the technical terms are used only for explaining specific exemplary embodiments while not limiting the present invention. The terms of a singular form may include plural forms unless otherwise specified. The meaning of “include,” “comprise,” “including,” or “comprising,” specifies a property, a region, a fixed number, a step, a process, an element and/or a component but does not exclude other properties, regions, fixed numbers, steps, processes, elements and/or components.

Additionally, the embodiments in the detailed description will be described with sectional views or plan views as ideal exemplary views of the present invention. In the drawings, the dimensions of layers and regions are exaggerated for clarity of illustration. Areas exemplified in the drawings have general properties, and are used to illustrate specific shapes of device regions. Thus, these should not be construed as limiting to the scope of the present invention.

In an exemplary embodiment, a blood is exemplified as a fluid containing a target material, to which the present invention is not limited. Other body fluids (e.g., urine and saliva) containing a target material may be used to diagnose a target material.

In the specification, a target material is a biomaterial showing a specific nature, which is interpreted as having the same meaning as target molecules, assays, or analytes. In an exemplary embodiment, a biomaterial may be an antigen.

In the specification, a sensing material is a biomaterial forming a specific binding to a target material, which is interpreted as having the same meaning as probe molecules, receptors, or acceptors. In an exemplary embodiment, a sensing material may be an antibody.

Hereinafter, a disposable diagnostic kit according to an exemplary embodiment of the present invention will be described in detail with reference to the accompanying drawings.

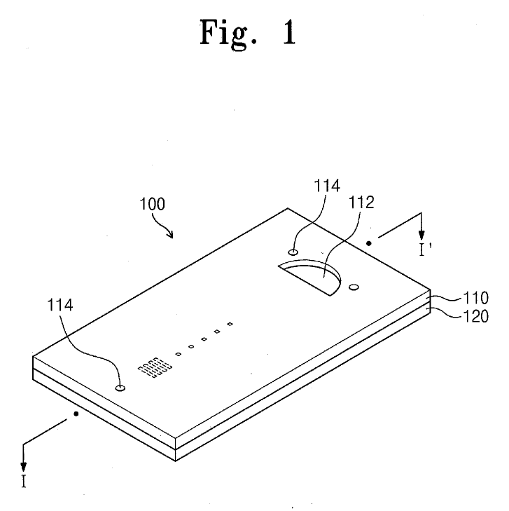

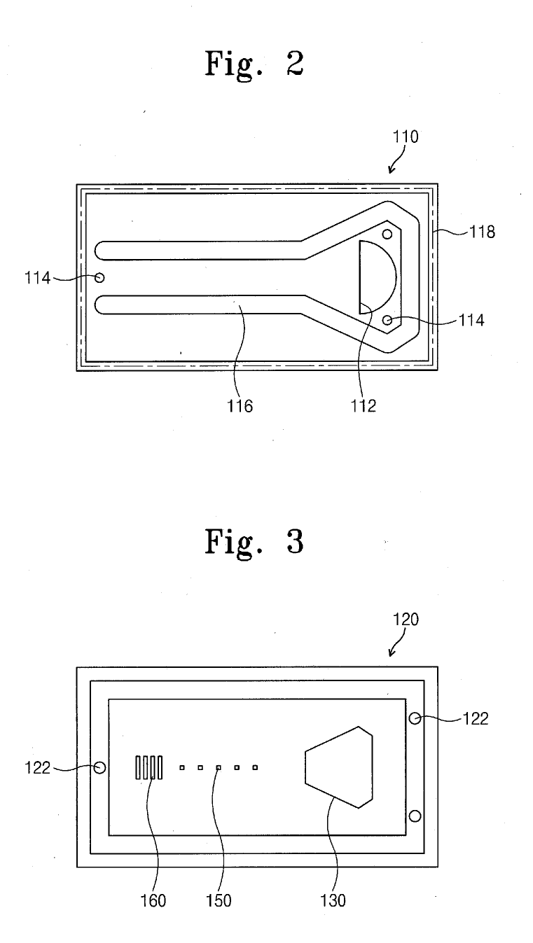

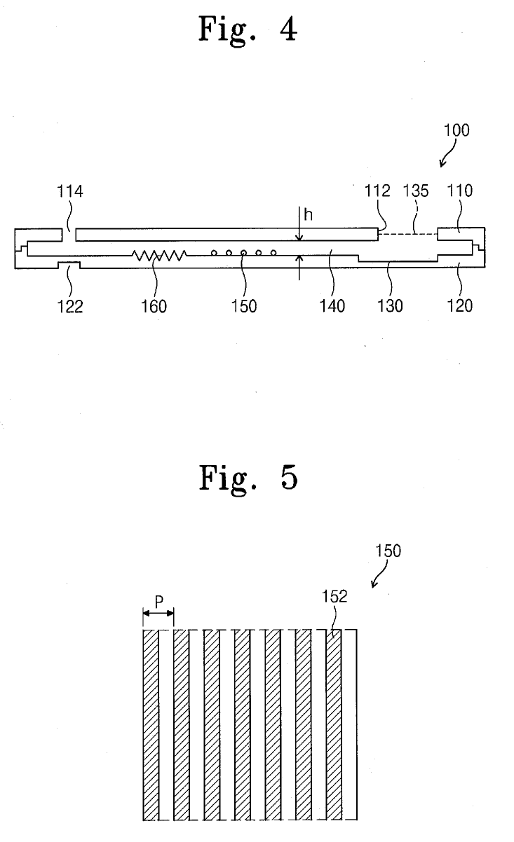

FIG. 1 is a perspective view of a disposable diagnostic kit according to an exemplary embodiment of the present invention. FIG. 2 is a view showing a top plate of the disposable diagnostic kit according to an exemplary embodiment of the present invention. FIG. 3 is a view showing a bottom plate of the disposable diagnostic kit according to an exemplary embodiment of the present invention. FIG. 4 is a cross-sectional view taken along a line I-I′ of FIG. 1. FIG. 5 is a view showing a target material reactor of the disposable diagnostic kit according to an exemplary embodiment of the present invention.

Referring to FIGS. 1 to 5, a disposable diagnostic kit 100 according to an exemplary embodiment of the present invention includes a top plate 110, a bottom plate 120, a preprocessor 130/135, a microfluidic channel 140, a target material reactor 150, and a fluid supply controller 160.

Referring to FIG. 1, the disposable diagnostic kit 100 may be fabricated by coupling the top plate 110 and the bottom plate 120 together. The top plate 110 and the bottom plate 120 may be formed of a transparent material capable of transmitting light. For example, the top plate 110 and the bottom plate 120 may be plastic or glass substrates. Also, the top plate 110 and the bottom plate 120 may be transparent oxide substrates formed of a silicon nitride (SiN), a titanium oxide (TiO₂), a tantalum oxide (Ta₂O₅), or an aluminum oxide (Al₂O₃). In other words, the top plate 110 and the bottom plate 120 may be formed of a material having a high index of refraction. Also, the top plate 110 and the bottom plate 120 may be formed of a transparent polymer such as polydimethylsiloxane (PDMS), polymethylmethacrylate (PMMA), polycarbonate (PC), cyclic olefin copolymer (COC), polyamide (PA), polyethylene (PE), polypropylene (PP), polyphenylene ether (PPE), polystyrene (PS), polyoxymethylene (POM), polyetheretherketone (PEEK), polytetrafluoroethylene (PTFE), polyvinylchloride (PVC), polyvinylidene fluoride (PVDF), polybutyleneterephthalate (PBT), fluorinated ethylenepropylene (FEP), or perfluoralkoxyalkane (PFA).

Referring to FIGS. 1 to 4, the top plate 110 of the disposable diagnostic kit 100 has a fluid inlet 112 formed to inject a body fluid containing target materials. The fluid inlet 112 pierces the top plate 110, and transmits the body fluid directly to the preprocessor 130/135. Accordingly, the fluid inlet 112 of the top plate 110 is formed corresponding to the preprocessor 130/135 of the bottom plate 120.

A predetermined region of the top plate 110 may have at least one air outlet 114 formed therein. The air outlet 114 discharges air from the microfluidic channel 140 so that the body fluid injected through the fluid inlet 112 can flow smoothly through the microfluidic channel 140. Also, the top plate 110 may have a bonding member 116 bonded to the bottom plate 120. That is, the air outlet 114 may be connected to the microfluidic channel 140 formed by the top and bottom plates 110 and 120.

Also, an edge of the top plate 110 may have a coupling line 118 formed to be coupled to the bottom plate 120. The coupling line 118 is formed so that the edges of the top and bottom plates 110 and 120 can be coupled together. The top and bottom plates 110 and 120 may be completely coupled by ultrasonic welding. For example, the coupling line 118 serves as a welding line that is used for ultrasonic welding of the top and bottom plates 110 and 120. The welding line may be formed in the shape of a triangular pyramid and groove.

The bottom plate 120 of the disposable diagnostic kit 100 has the preprocessor 130/135, the microfluidic channel 140, the target material reactor 150, and the fluid supply controller 160 formed therein.

The preprocessor 130/135 selects only target materials reacting or coupling with a sensing material, from a body fluid containing various target materials. That is, the preprocessor 130/135 filters (or selects) a body fluid containing a target material to be detected. For example, the preprocessor 130/135 removes hemocytes (i.e., unnecessary components) from a blood.

Examples of the body fluid include blood, urine, and saliva. The body fluid may contain not only a target material to be detected, but also nonspecific molecules not coupling with sensing materials.

The body fluid may contain various target materials, and it is necessary to remove unnecessary target materials from the body fluid in order to accurately and rapidly detect a specific target material to be diagnosed among the target materials.

For example, the body fluid contains various hemocytes and plasmas, and contains protein components such as various cells, lipid, catabolite, moisture, enzyme, antigen, and antibody. The specific target material to be detected is mainly present in the plasma. Examples of the target material include protein, nucleic acid, virus, cell, organic molecule, and inorganic molecule. The protein molecule may be any biomolecules such as antigen, antibody, matrix protein, enzyme, and coenzyme. The nucleic acid may be DNA, RNA, PNA, LNA, or a mixture thereof.

The preprocessor 130/135 includes a fluid storage chamber 130 and a micro filter 135. The fluid storage chamber 130 has a bottom surface formed lower than the level of the bottom plate 120, and stores a body fluid containing a target material. The micro filter 135 is installed between the fluid storage chamber 130 and the fluid inlet 112 of the top plate 110. The micro filter 135 filters off hemocytes from a body fluid, and passes only a plasma containing a target material into the fluid storage chamber 135. For example, the micro filter 135 may be a micro paper filter having micro holes formed therein. The thickness of the micro paper filter and the sizes of the micro holes may vary depending on the sizes of target materials contained in a body fluid, or the amount of a body fluid flowing into the preprocessor 130/135.

The blood filtered by the preprocessor 130/135 may move through the microfluidic channel 140 to the target material reactor 150. The microfluidic channel 140 moves the blood filtered by the preprocessor 130/135 to the target material reactor 150. The microfluidic channel 140 is formed by coupling the bottom of the top plate 110 with the top of the bottom plate 120 in such a way that they are spaced apart from each other by a predetermined distance. Herein, the distance between the top and bottom plates 110 and 120 is controlled to generate a sufficient capillary force. By the capillary force, the preprocessed blood can pass the microfluidic channel 140. For example, the microfluidic channel 140 may have a diameter or height ‘h’ of about 1 nm to about 40 μm. Also, the microfluidic channel 140 may be hydrophilically surface-treated so that the preprocessed blood can move smoothly.

The target material reactor 150 biochemically reacts or couples sensing materials with target materials to be detected. Without labeling material, light is irradiated onto the target material reactor 150 to detect if there is a biochemical reaction or coupling between a sensing material and a specific target material. The target material reactor 150 may be a resonance reflection filter that measures a wavelength change of light by the biochemical reaction or coupling between target materials and sensing materials to detect a specific target material. The resonance reflection filter uses the peak of a reflection spectrum created by diffraction gratings that can serve as a high-refractive waveguide. The reflection spectrum, which is created by a coupling with a mode where the light diffracted by the diffraction gratings is guided through the high-refractive waveguide, is narrow in linewidth, thus making it possible to implement a high-resolution biosensor.

Referring to FIG. 5, the target material reactor 150 includes nanopatterns 152 that generate resonant reflected light. Light penetrated into the nanopatterns 152 or reflected by the nanopatterns 152. And, a wavelength of light penetrated into the nanopatterns 152 or a wavelength of light reflected by the nanopatterns 152 varies depending on the reaction between the sensing materials and the target materials. The number of the nanopatterns 152 may be determined according to the amount or number of sensing materials for a disease or a symptom to be diagnosed. The nanopatterns 152 may be formed through a photolithography process, an electron-beam lithography process, or an imprint process that transfers nanopatterns using a stamp. Also, the disposable diagnostic kit 100 including the nanopatterns 152 may be formed through an injection molding process. The injection molding was carried out by using a metal mold having nano patterns for target material reactor 150. Therefore, it is possible to mass produce the disposable diagnostic kit 100. For example, the nanopatterns 152 may be a periodically-repeated line-and-space pattern that generates resonant reflected light such as the 780nm band. The nanopatterns 152 may be formed in a square region, and the period ‘p’ and arrangement of the nanopatterns 152 may vary depending on the wavelength of desirable resonant reflected light.

Sensing materials, which react or couple with specific target materials of a disease or a symptom to be diagnosed, are immobilized on the surfaces of the nanopatterns 152 of the target material reactor 150. The sensing materials may be protein, cell, virus, nucleic acid, organic molecule, and inorganic molecule, according to the target material to be detected. The protein may be any target materials such as antigen, antibody, matrix protein, enzyme, and coenzyme. The nucleic acid may be DNA, RNA, PNA, LNA, or a mixture thereof.

The sensing materials may be immobilized on the surfaces of the nanopatterns 152 by chemical adsorption, covalent-binding, electrostatic attraction, co-polymerization, or avidin-biotin affinity system.

That is, the sensing materials may be immobilized on the surfaces of the nanopatterns 152 directly or indirectly by using organic molecules as intermediate medium molecules. Also, a functional group may be induced on the surfaces of the nanopatterns 152 in order to immobilize the target materials on the nanopatterns 152. For example, functional groups, such as a carboxyle group (—COOH), a thiol group (—SH), a hydroxyl group (—OH), a silane group, an amine group, and an epoxy group, may be induced on the surfaces of gold nanoparticles.

Also, a space between the nanopatterns 152 may be blocked so that the sensing materials are not immobilized therein.

Also, the fluid supply controller 160 may be formed on the bottom plate 120 of the disposable diagnostic kit 100 to control the supply rate of the blood supplied to the target material reactor 150.

That is, the fluid supply controller 160 serves as a time gate that delays the flow of a preprocessed blood. Accordingly, the fluid supply controller 160 enables a sensing material and a specific target material to react with each other for a sufficient time.

The fluid supply controller 160 may be formed by modifying the shape of the microfluidic channel 140 through which a blood flows. That is, the fluid supply controller 160 may control the fluid supply by changing the sectional area of the microfluidic channel 140. For example, the fluid supply controller 160 may include microgrooves formed at the bottom plate 120. The size of number of the microgrooves may vary depending on the reaction times according to the sensing material and the specific target materials. Also, the microgrooves may be surface-treated with hydrophobic materials.

The fluid supply controller 160 including the microgrooves locally increases the diameter of the microfluidic channel 140, thereby making it possible to reduce the capillary force of the microfluidic channel 140. Accordingly, the flow rate of the blood flowing through the microfluidic channel 140 can be reduced.

That is, the disposable diagnostic kit 100 includes a region where the distance between the top and bottom plates 110 and 120 is equal to ‘h’ (i.e., a region of the microfluidic channel 140), and a region where the distance between the top and bottom plates 110 and 120 is greater than ‘h’ (i.e., a region of the fluid supply controller 160).

Also, as illustrated in FIG. 4, at least one alignment groove 122 may be formed in a predetermined region of the bottom plate 120, which is to be aligned with or mounted on a reader (not shown) for detecting a resonant reflected light generated by the target material reactor 160 when a specific target material is detected. In another embodiment, at least one alignment groove 122 may be formed at the top plate 110.

The reader may detect a target material by measuring the wavelength of a resonant reflected light before/after the coupling or reaction of the target material with sensing materials.

As described above, the present invention uses plastic materials to form the top and bottom plates, thus making it possible to provide an inexpensive disposable diagnostic kit.

The disposable diagnostic kit of the present invention includes the preprocessor, thus making it possible to directly inject a biomaterial detecting sample (i.e., a blood) into the diagnostic kit without preprocessing. Therefore, the present invention can detect/analyze a biomaterial rapidly. Also, the present invention can detect a biomaterial in a label-free fashion without limitation on the environmental conditions for detection of the biomaterial.

That is, the present invention performs fluid movement and biomaterial detection in the single diagnostic kit, thus making it possible to diagnose a disease more simply.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A disposable diagnostic kit comprising:
a preprocessor filtering target materials from a fluid containing various biomaterials;
a target material reactor comprising a diffraction grating on whose surface sensing materials reacting with the target materials are immobilized, wherein a wavelength of light penetrated into the diffraction grating or a wavelength of light reflected by the diffraction grating varies depending on the target materials; and
a microfluidic channel moving the filtered fluid from the preprocessor to the target material reactor.

2. The disposable diagnostic kit of claim 1, further comprising a fluid supply controller controlling the supply rate of the fluid moving through the microfluidic channel.

3. The disposable diagnostic kit of claim 2, wherein the disposable diagnostic kit is fabricated by coupling a top plate and a bottom plate together in such a way that the facing planes of the top and bottom plates are spaced apart from each other by a predetermined distance.

4. The disposable diagnostic kit of claim 3, wherein the microfluidic channel is formed by the facing planes of the top and bottom plates.

5. The disposable diagnostic kit of claim 2, wherein the fluid supply controller changes the sectional area of the microfluidic channel to control the supply rate of the fluid.

6. The disposable diagnostic kit of claim 5, wherein the fluid supply controller comprises a plurality of grooves formed at the top or bottom plate to change the distance between the top and bottom plates.

7. The disposable diagnostic kit of claim 3, wherein the top plate comprises a fluid inlet configured to provide the fluid containing the target materials directly to the preprocessor.

8. The disposable diagnostic kit of claim 3, wherein the top plate comprises an air outlet configured to discharge air from the microfluidic channel so that the fluid is moved by a capillary force.

9. The disposable diagnostic kit of claim 3, wherein the bottom plate has an alignment groove aligned with a reader that measures a wavelength of light penetrated into the diffraction grating or a wavelength of light reflected by the diffraction grating.

10. The disposable diagnostic kit of claim 3, wherein the top and bottom plates have a coupling line formed along an edge thereof where the top and bottom plates are coupled together.

11. The disposable diagnostic kit of claim 3, wherein the top and bottom plates are formed of transparent materials penetrating or reflecting light.

12. The disposable diagnostic kit of claim 1, wherein the diffraction grating of the target material reactor comprises a plurality of nanopatterns.

13. The disposable diagnostic kit of claim 12, wherein the nanopatterns are periodically formed, and the light wavelength varies depending on the period of the nanopatterns.

14. The disposable diagnostic kit of claim 1, wherein the sensing materials are immobilized by a carboxyle group (—COOH), a thiol group (—SH), a hydroxyl group (—OH), a silane group, an amine group (—NH₂), or an epoxy group induced on the surface of the diffraction grating.

15. The disposable diagnostic kit of claim 14, wherein the reaction between the sensing materials and the target materials includes nucleic acid hybridization, antigen-antibody reaction, or enzyme conjugation.

16. The disposable diagnostic kit of claim 1, wherein the fluid containing the target materials includes a blood, and the preprocessor passes plasma from the blood.

17. The disposable diagnostic kit of claim 1, wherein the preprocessor, the microfluidic channel, the target material reactor, and the fluid supply controller has a hydrophilic surface.

18. The disposable diagnostic kit of claim 1, wherein the target materials comprise at least one of nucleic acid, cell, virus, protein, organic molecule, and inorganic molecule.

19. The disposable diagnostic kit of claim 18, wherein the nucleic acid comprises at least one of DNA, RNA, PNA, LAN, and a mixture thereof.

20. The disposable diagnostic kit of claim 18, wherein the protein comprises at least one of enzyme, matrix, antigen, antibody ligand, aptamer, and receptor.
